(19) United States
(12) Patent Application Publication
(10) Pub. No.: US 2025/0259516 A1
(43) Pub. Date: Aug. 14, 2025

(54) SENSOR ARRAYS WITH INTEGRATED READOUT

(71) Applicant: Tacta Systems Inc., Palo Alto, CA (US)

(72) Inventors: Andreas Bibl, Los Altos, CA (US); Dariusz Golda, Portola Valley, CA (US); Nahid Harjee, Sunnyvale, CA (US); Patrick M. Smith, San Jose, CA (US)

(21) Appl. No.: 18/666,203

(22) Filed: May 16, 2024

Related U.S. Application Data
(60) Provisional application No. 63/553,095, filed on Feb. 13, 2024.

(51) Int. Cl. G08B 6/00

(52) U.S. Cl. CPC G08B 6/00

(57) ABSTRACT

A sensing system may include a sensor array comprised of a plurality of sensors coupled with a flexible circuit. Each sensor may include a sensing element and an analog-to-digital converter die coupled with the sensing element. The sensing element may include a sensor area coupled with a sensor die to generate an analog output based on sensing, and the analog-to-digital converter die may include circuitry to perform analog-to-digital conversion of the analog output to generate a digital output. The sensing system may further include a controller connected to the sensor array. The controller can cause a sensor of the sensor array to transmit a digital output that indicates sensing by the sensor in response to receiving a digital input. Other aspects are also described and claimed.

### RELATED APPLICATIONS

This patent application claims the benefit of priority of U.S. Provisional Application No. 63/553,095, filed Feb. 13, 2024, which is incorporated herein by reference in its entirety.

### BACKGROUND

### Field

This disclosure relates generally to sensor arrays and, more specifically, to sensor arrays with integrated readout. Other aspects are also described.

### Background Information

A sensor array may refer to a group of sensors used for collecting information about an environment. Sensors of a sensor array may be arranged in a certain geometric configuration or pattern. Sensor arrays may enable collecting information over a greater area than a single sensor, and in multiple dimensions of the environment.

In operation, a sensor of a sensor array can generate an output signal indicating detection of a physical phenomenon. For example, a piezoelectric sensor can utilize the piezoelectric effect to detect changes in pressure, acceleration, temperature, strain, or force by converting such detections to an electrical charge. In another example, a capacitive sensor can utilize capacitive sensing to detect an object in proximity that may be conductive or may have a dielectric constant that is different from air.

### SUMMARY

Implementations of this disclosure include utilizing sensors of a sensor array that can each perform analog sensing and conversion to a digital output at the point of sensing to enable reduced wiring and/or selective readout of sensors. In some implementations, a sensing system may include a sensor array comprised of a plurality of sensors coupled with a flexible circuit. Each sensor of the array may include a sensing element and an analog-to-digital converter die coupled with the sensing element. The sensing element may include a sensor area coupled with a sensor die to generate an analog output based on sensing in the environment. The analog-to-digital converter die may include circuitry to perform analog-to-digital conversion (ADC) of the analog output to generate a digital output. The sensing system may further include a controller connected to the sensor array. The controller can cause a sensor of the sensor array to transmit a digital output that indicates sensing by the sensor in response to receiving a digital input. Other aspects are also described and claimed.

The above summary does not include an exhaustive list of all aspects of the present disclosure. It is contemplated that the disclosure includes all systems and methods that can be practiced from all suitable combinations of the various aspects summarized above, as well as those disclosed in the Detailed Description below and particularly pointed out in the Claims section. Such combinations may have particular advantages not specifically recited in the above summary.

### BRIEF DESCRIPTION OF THE DRAWINGS

Several aspects of the disclosure herein are illustrated by way of example and not by way of limitation in the figures of the accompanying drawings in which like references indicate similar elements. It should be noted that references to “an” or “one” aspect in this disclosure are not necessarily to the same aspect, and they mean at least one. Also, in the interest of conciseness and reducing the total number of figures, a given figure may be used to illustrate the features of more than one aspect of the disclosure, and not all elements in the figure may be required for a given aspect.

FIG. 1 is an example of a sensing system including a plurality of sensor arrays.

FIG. 2 is a first example of a cross-section of a sensor of a sensor array.

FIG. 3 is an example of a top view of a plurality of sensors of a sensor array.

FIG. 4 is a first example of a controller reading digital data from a sensor array.

FIG. 5 is a second example of a controller reading digital data from a sensor array.

FIG. 6 is a third example of a controller reading digital data from a sensor array.

FIG. 7 is a flowchart of an example of a process for reading sensors of a sensor array.

### DETAILED DESCRIPTION

When utilizing sensor arrays, it is often desirable to have a high resolution of sensing. This may involve many sensors in the array (e.g., to obtain more points of sensing) and/or a high density of sensors in the array (e.g., more sensors per unit area). For example, it may be desirable to have thousands of sensors or more in a relatively limited space, such as a sensing glove. However, increasing the number of sensors of a sensor array can complicate readout of the sensors by a controller. For example, when many sensors are present, it may be difficult for the controller to read all the sensors fast enough to detect changes occurring in the environment. This may be especially problematic when piezoelectric sensors that are susceptible to sensing voltage decay are used. Also, when a limited space is involved, such as a sensing glove, it may be difficult to route wiring to all the sensors to enable the readout. As a result, sensor arrays may be limited to a lower resolution and/or larger areas.

Implementations of this disclosure address problems such as these by utilizing sensors of a sensor array that can each perform analog sensing and conversion to a digital output at the point of sensing to enable reduced wiring and/or selective readout of sensors. Based on the sensors, wiring, and/or configurations of the sensing system as described herein, a controller can target one or more sensors in one or more sensor arrays to read digital data from the one or more sensors, with each element of sensed information representing a digital capture of analog sensing performed by a sensor (e.g., the force, temperature, or proximity sensing in the environment).

The sensors described herein may each include a sensing element coupled with an analog-to-digital converter die. The sensing elements may additionally include a sensor area coupled with a sensor die, which can include implementing circuitry coupled with the sensor area of the analog-to-digital converter die.

In some implementations, a sensing system may include a sensor array comprised of a plurality of sensors, such as 1,000 sensors, 10,000 sensors, or more, coupled with a flexible circuit. The sensor array may be integrated with a material that could have relatively limited space or a small form factor, such as an article worn by a user (e.g., a sensing glove). Each sensor of the array may include a sensing element (e.g., a force, temperature, or proximity sensing element) and an analog-to-digital converter die (e.g., a metal oxide semiconductor field effect transistor (MOSFET) or (CMOS) integrated circuit (IC) implementing circuitry, such as a charge amplifier and an ADC) coupled with the sensing element. The sensing element may comprise a sensor area (e.g., a piezoelectric or capacitive sensor) coupled with a sensor die (e.g., another IC that implements circuitry). The sensing element may generate an analog output, based on sensing in the environment, sent to the analog-to-digital converter die. The analog-to-digital converter die, in turn, may utilize circuitry to perform ADC of the analog output to generate a digital output.

The sensing system may further include a controller connected to the sensor array. The controller can cause a sensor of the sensor array to transmit a digital output that indicates sensing at a given time by the sensor in response to receiving a digital input. The controller can utilize a communications device to send the sensed information to another system. As a result, the sensing system enables a selective readout of one or more sensors of a sensor array, performed relatively fast and with high resolution of the array, with a high density of sensors in the array.

In some implementations, a sensing element (e.g., a sensor area coupled with a sensing die) may be combined with a mixed signal die (e.g., an analog-to-digital converter die) to convert a sensor's analog signals into a digital representation (e.g., 8 bits, 12 bits, or more) in a single integrated device. The sensor may be coupled with a metal wire-pattern flexible or rigid circuit substrate. This may enable the circuit to connect a plurality of sensors with reducing complexity/wiring. For example, the analog-to-digital converter die could be coupled with flexible circuit wiring implemented by a flexible substrate. A plurality of through vias, such as through-silicon-vias (TSVs), and connections through the analog-to-digital converter die, from its front surface to its back surface, may couple with the sensing element above and the flexible circuit wiring below the analog-to-digital converter die.

In some implementations, a sensor die may be bonded to an analog-to-digital converter die with a sensor area exposed on a top of the sensor. A plurality of through vias and contacts may be included through the sensor die, from its front surface to its back surface, forming electrical connections with the analog-to-digital converter die below. Bonding between the sensor die and the analog-to-digital converter die could be performed, for example, at the die level with a pick-and-place process, or at the wafer level after thinning and bumping (or electrical contacts exposure). Thinning may be performed, for example, by grinding, dry etching, wet etching (e.g., allowing etch stop selectivity against a different material layer in the die), or electrochemical etching (e.g., electrically contacting n or p wells in the silicon wafer to provide etch selectivity/stopping).

In some implementations, the sensor die may be inverted and bonded face-to-face with the analog-to-digital converter die (e.g., top electrical contacts of the sensor die may directly contact top electrical contacts of the analog-to-digital converter die). This may enable eliminating through vias of the sensor die. In some cases, a cavity may be etched through the substrate of the sensor die to expose a bottom of the sensing element to the environment to further improve sensitivity.

In some implementations, the sensor may be pressed or embedded into a flexible circuit substrate so that a top of the analog-to-digital converter die is flush with the flexible circuit substrate's top surface. This may enable electrical contacts and wiring of the flexible circuit to be overlaid to contact an upper part of the sensor, as opposed to a bottom of the sensor. For example, the sensor die may include through vias extending through its thickness to contact the analog-to-digital converter die, and the analog-to-digital converter can eliminate through vias altogether based on the configuration.

In some implementations, sensors may be connected in series. Electrical signals between the sensors can be reduced by having the sensors share a same basic set of signal wires, such as V_{DD} (power), ground, clock input, select (e.g., digital input), output (e.g., digital output), and/or V_{SS} (power). A pulse of the digital input can trigger a first sensor's measurement. After that measurement is performed, with data from the sensor sent via the digital output, the first sensor can trigger a second sensor to transmit its measurement, and so forth. The controller can therefore selectively cause the digital inputs to be transmitted to the sensors and can read the digital data from the digital outputs.

In some implementations, the controller may be a local controller that performs processing before sending measurement data from sensors to another controller, such as a global controller. The global controller can provide external communications via wired or wireless connections to another system (e.g., USB, LVDS, SPI, Bluetooth, Ethernet, etc.). In some implementations, additional signals may be used for the control and/or synchronization of the readout performed by the controller.

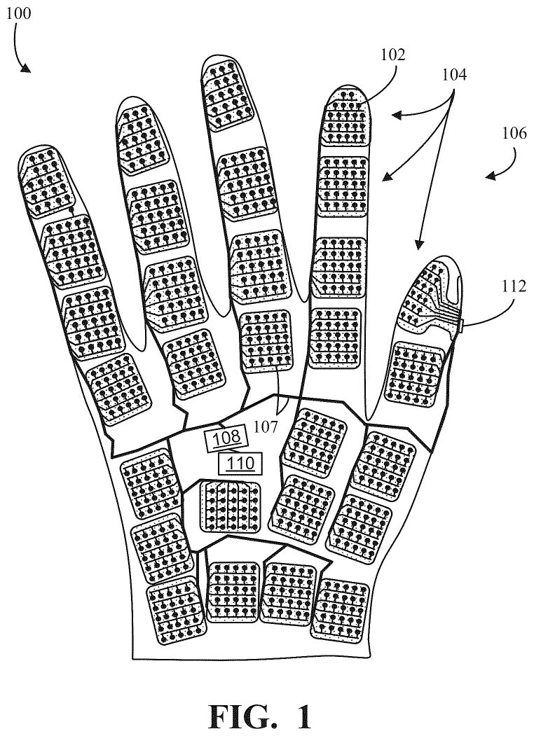

FIG. 1 is an example of a sensing system 100 including a plurality of sensors 102 of a plurality of sensor arrays 104 coupled with a flexible circuit. The sensing system 100 may perform an integrated readout of sensors 102 as described herein. The sensor arrays 104 may implement many sensors while being integrated with an article 106 which may be deformable and/or have relatively limited space. For example, the sensing system 100 may be a wearable system that is integrated with a sensing glove worn by a user. The sensor arrays 104 may include, for example, 1,000 sensors, 10,000 sensors, or more, integrated with the article 106. Each sensor array 104 may correspond to a group of sensors 102 arranged in a location of the article 106. For example, a first sensor array 104 may correspond to a first group of 10 sensors, 100 sensors, or more, arranged at a first finger or fingertip of the sensing glove, a second sensor array 104 may correspond to a second group of 10 sensors, 100 sensors, or more, arranged at a second finger or fingertip of the sensing glove, and so forth.

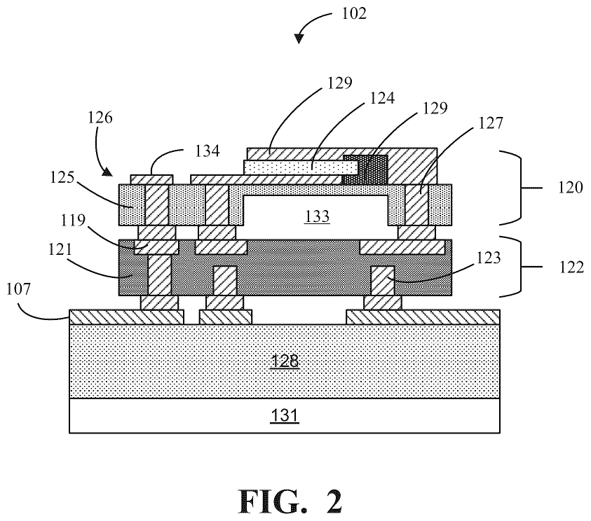

With additional reference to FIG. 2, each sensor 102 may include a sensing element 120 coupled with an analog-to-digital converter die 122 (e.g., a microfabricated IC). In various configurations, the sensing element 120 could be a force, temperature, or proximity sensing element 120. The sensing element 120 may include a microfabricated sensor area 124 (e.g., for sensing a normal force, shear force, temperature, and/or proximity) coupled with a sensor die 126. In some implementations, the sensor area 124 may comprise a piezoelectric, capacitive, or other sensor. The sensor die 126 could be an IC that implements circuitry coupled with the sensor area 124 on a first side and with the analog-to-digital converter die 122 on a second side. For example, the sensor die 126 could implement circuitry to connect the sensor area 124 to the analog-to-digital converter die 122. The sensing element 120 may generate an analog output based on sensing (e.g., the sensing glove touching an object), and the sensor die 126 can transmit the analog output to the analog-to-digital converter die 122. In some cases, a cavity 133 may optionally be formed in the base substrate 125 of the sensor die 126 to enable flex of the sensor die 126 with an application of force directed to the sensor area 124. This may further improve sensitivity.

In the exemplary implementation illustrated, the sensor die 126 can include a base substrate 125. Where the IC implementing circuitry is included, the base substrate 125 may be silicon or a III-V semiconductor, for example, with the IC implementing circuitry and back-end-of-the-line (BEOL) routing 134 formed using customary techniques. As shown, the BEOL routing 134 can include landing pads, for example, for external connection, as well as routing for connection with the sensor area 124 and through vias 127. As shown, a plurality of through vias 127 can extend through the base substrate 125 of the sensor die 126 to provide vertical interconnection to the analog-to-digital converter die 122. In a particular implementation, the through vias 127 can be TSVs where the base substrate 125 is silicon. A plurality of leads 129 may be further connected with the sensor area 124, and electrically connected with the working circuitry of the sensor die 126 and/or the through vias 127.

The sensing element 120 may provide an analog signal indicating a measurement from sensing, such as a measurement of a normal force, shear force, temperature, or proximity of an object, detected via the sensor area 124. The sensing element 120 may be relatively small, having lateral dimensions, for example, in a range of 100 to 1000 μm, or more specifically, 100 to 300 μm, per side, and a thickness in a range of 100 to 1000 μm, or more specifically, 100 to 300 μm. Bottom side electrical connections of the sensing element 120 may be connected to (e.g., bonded to) corresponding electrical connections on a top side of the analog-to-digital converter die 122 for example, with solder bumps, metal-metal bonds, or other suitable electrically conductive bonding material. The sensing element 120 may be bonded to or fabricated with the analog-to-digital converter die 122 (e.g., in direct electric contact) with the sensor area 124 exposed on a top of the sensor 102. In this way, the analog-to-digital converter die 122 may provide power and ground to the sensing element 120 and may amplify and convert analog signals from the sensing element 120 at the exact location of the sensor 102 in the sensor array 104.

Bonding between the sensor die 126 and the analog-to-digital converter die 122 could be performed, for example, at the die level with a pick-and-place process, or at the wafer level followed by singulation. Stacking the sensing element 120 on the analog-to-digital converter die 122 for a given sensor 102 can facilitate integration of a greater number of sensors 102 per unit area in the sensing system 100. Additionally, this may enable more available area for strain relief cutouts to allow greater deformability of the article 106, including as described with respect to FIG. 4.

The analog-to-digital converter die 122 may include circuitry to perform amplification and ADC of the analog output, from the sensing element 120, to generate a digital representation of the analog output. For example, the analog-to-digital converter die 122 could implement a charge amplifier to amplify voltages and/or currents, generated by the sensing element 120, for the ADC. The digital output could comprise 8 bits, 12 bits, or more, representing the sensing. In the exemplary implementation illustrated the analog-to-digital converter die 122 can include a base substrate 121. Where the IC implementing circuitry is included, the base substrate 121 may be silicon or a III-V semiconductor, for example, with the IC implementing circuitry and back-end-of-the-line routing 119 formed using customary techniques. As shown, through vias 123 can extend through the base substrate 121 of the analog-to-digital converter die 122 to provide vertical interconnection between the sensing element 120 and electrical interconnects 107 (e.g., copper wiring) over the flexible circuit 128 (or sensor arrays 104 placed or formed thereon), which can optionally be supported by a substrate 131 (e.g., glass, polymer) that can be adhered to the article 106 on a bottom side. In a particular implementation, the through vias 123 can be TSVs where the base substrate 121 is silicon.

The analog-to-digital converter die 122 could be a MOSFET or CMOS IC comprised of single-crystalline silicon. Similar to the sensor die 126, the analog-to-digital converter die 122 may have lateral dimensions, for example, in a range of 100 to 1000 μm, or more specifically, 100 to 300 μm, per side, and a thickness in a range of 100 to 1000 μm, or more specifically, 100 to 300 μm.

In some implementations, the analog-to-digital converter die 122 may be coupled with the flexible circuit 128 by conventional pick-and-place mounting methods (e.g., flip-chip solder bonding). The sensing element 120 and the analog-to-digital converter die 122 may be micro-fabricated separately from the flexible circuit 128 and subsequently assembled to the flexible circuit 128 (which may be coupled with the article 106). In some implementations, each sensor array 104 may be coupled with its own flexible circuit 128. The small footprint of the sensor die 126 and the analog-to-digital converter die 122 may enable a small radius of curvature directly under and adjacent to the sensor 102. The analog-to-digital converter die 122 may be coupled with electrical interconnect 107 (e.g., copper wiring) of the sensor array 104 that may, in turn, be coupled with other components of the sensing system 100.

Referring again to FIG. 1, the sensing system 100 may include a controller 108 (another IC, such as an application-specific integrated circuit (ASIC) or field-programmable gate array (FPGA)) connected to sensors 102 of the plurality of sensor arrays 104 and to a communications device 110. For example, the sensors 102 could be on a palmar side of a sensing glove, and the controller 108 and the communications device 110 could be on the palmar side or a dorsal side of the sensing glove. The controller 108 may connect directly and/or indirectly to the sensors 102. For example, in some cases, the controller 108 may connect directly to sensors 102, and in other cases, the controller 108 may be a global controller connected to one or more local controllers that are connected to the sensors 102. For example, the controller 108 could connect to a local controller 112 (e.g., another IC, such as an ASIC or FPGA) arranged on a section of the article 106 (e.g., a dorsal side of a thumb of the sensing glove). The local controller 112, in turn, may connect to sensors 102 of one or more sensor arrays 104 in the section (e.g., the thumb). The local controller 112 can process digital outputs from sensors 102 in the section to generate a compressed bitstream for the controller 108. In some implementations, the controller 108 may be a hybrid controller operating as both a global controller (e.g., connected to local controllers arranged in some sections of the article 106) and a local controller (e.g., connected directly to sensors 102 in other sections of the article 106).

In operation, the controller 108 can cause one or more sensors 102 of one or more sensor arrays 104 to each transmit a digital output. A digital output form a sensor 102 may indicate sensing performed by that sensor 102 at a given time in response to receiving a digital input (e.g., a trigger). In some cases, the controller 108 can directly cause transmission of a digital output from a sensor 102, such as by sending a digital input to trigger a sensor 102. In other cases, the controller 108 can indirectly cause transmission of a digital output from a sensor 102, such as by causing a local controller to send a digital input to trigger a sensor 102, and/or by causing one sensor 102 to send a digital output to trigger another sensor 102.

The communications device 110 may enable transmission of a collection of digital data from sensors 102 to another system. The communications device 110 may utilize wired or wireless connections, such as universal serial bus (USB), low-voltage differential signaling (LVDS), serial peripheral interface (SPI), Bluetooth, or Ethernet, to transmit the digital data. For example, the controller 108 can receive digital outputs from the sensors 102 based on triggering those sensors, then utilize the communications device 110 to transmit a compressed bitstream encoding the digital outputs to another system, such as a host computer or server. As a result, the controller 108 can selectively perform readout of sensors 102 of sensor arrays 104 in the sensing system 100 to obtain sensing information relatively fast and with high resolution.

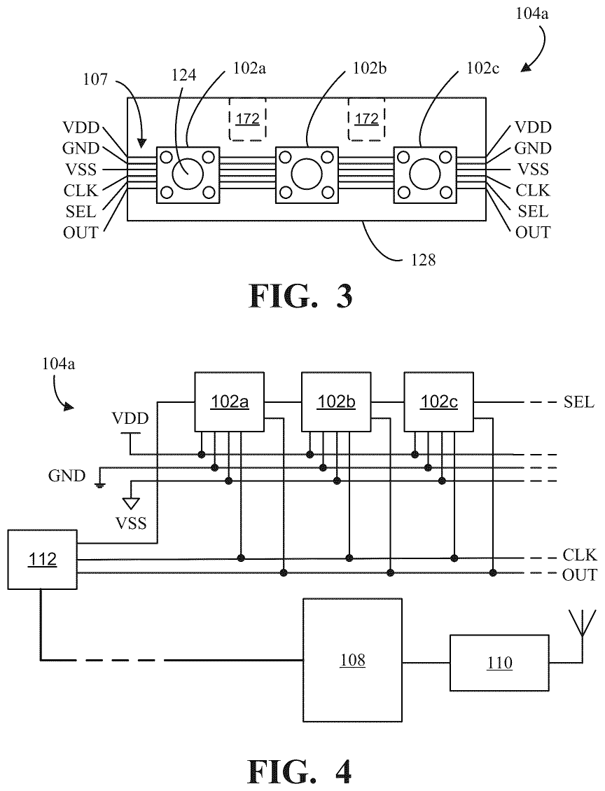

FIG. 3 is an example of a top view of sensors 102a-c of a sensor array 104a. For example, the sensor array 104a could be one of the plurality of sensor arrays 104 shown in FIG. 1. The sensors 102a-c may be connected in series (e.g., a daisy chain) via one or more signals of the electrical interconnect 107, such as a select line (“SEL”) or digital input. The number of electrical signals connected to the sensors 102a-c can be advantageously reduced by having the sensors 102a-c share a same basic set of signal wires, such as power 1 (“V_{DD}”), ground (“GND”), clock (“CLK”), the select line (“SEL”), a digital output (“OUT”), and/or power 2 (“V_{SS}”). The controller can utilize the select line to send the digital input to the sensors 102a-c and utilize the digital output to receive outputs from the sensors 102a-c. The controller can utilize the clock to synchronize the outputs from the sensors 102a-c during the readout. In some cases, additional signals may be used for further control and/or synchronization of the read out.

For example, with additional reference to FIG. 4, a controller (e.g., the controller 108 and/or the local controller 112) can cause the sensors 102a-c to each transmit a digital output indicating sensing in response to receiving a digital input (the select line). Initially, a pulse of the digital input from the controller can trigger the first sensor 102a to perform a measurement and generate a digital output that may be read by the controller. After that measurement is performed, with the digital output sent to the controller, the sensor 102a (operating as an upstream sensor) can then trigger sensor 102b (operating as a downstream sensor) to perform a next measurement and generate a next digital output that may be read by the controller. This process may continue as additional downstream sensors of the sensor array 104a receive digital inputs from upstream sensors to cause the downstream sensors to perform measurements and generate digital outputs. The controller can read the digital outputs from the sensors 102, sequentially, one after another, in the order of the sensors in the connected series.

In some implementations, the controller may be a local controller (e.g., the local controller 112) that triggers the sensors 102a-c. The local controller can then generate a first compressed bitstream, comprised of digital outputs from the sensors 102a-c, for a global controller (e.g., the controller 108). The global controller, in turn, can utilize the communications device 110 to send a second compressed bitstream comprised of the first compressed bitstreams from one or more local controllers in the sensing system 100.

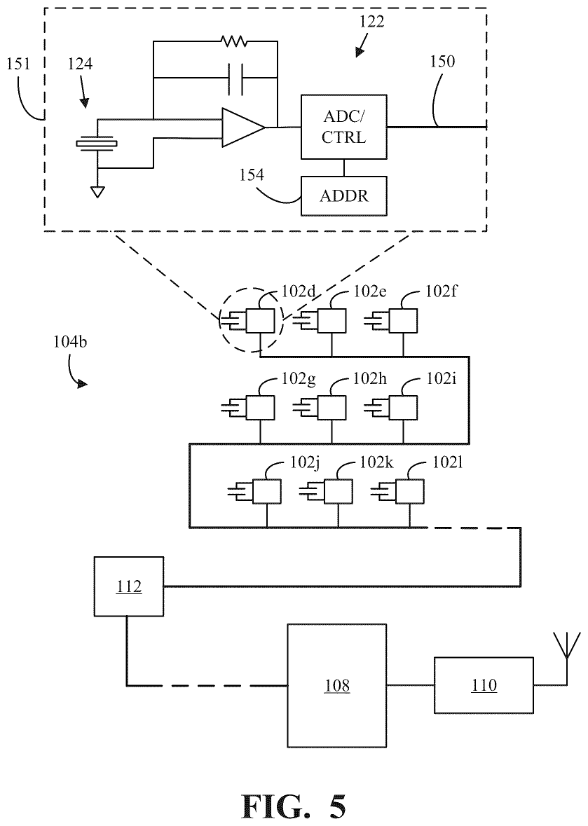

FIG. 5 is an example of a controller (e.g., the controller 108 and/or the local controller 112) that may utilize a serial bus 150 to read digital data from sensors 102d-1 of a sensor array 104b. For example, the sensor array 104b could be another of the plurality of sensor arrays 104 shown in FIG. 1. In the sensor array 104b, the controller can utilize the serial bus 150, connected to each sensor, to send a digital input to trigger the sensor and receive a digital output from the sensor. For example, the controller could individually target sensor 102e for readout by sending a digital input to sensor 102e and receiving a digital output from sensor 102e via the serial bus 150. The serial bus 150 could be, for example, an inter-integrated circuit (I²C) bus, a SPI bus, or a system management (SM) bus.

In some implementations, as shown in a detailed view 151, the sensing system 100 (e.g., the flexible circuit 128) may include address circuitry 154 that defines addresses of sensors of the sensor array. For example, the address circuitry 154 may define one unique address for sensor 102d, another unique address for sensors 102e, and so forth. The address circuitry 154 could comprise, for example, resistors (e.g., pull up or pull down resistors) that are placed, and/or fuses that are trimmed, on the flexible circuit 128. In some cases, the sensors may be individually programmed to define their addresses in the sensor array, such as by writing to a one-time programmable (OTP) register of each sensor (e.g., in addition or alternative to the address circuitry 154). This may enable the controller to uniquely identify and address sensors installed in given positions of the sensor array to selectively target the sensor for readout.

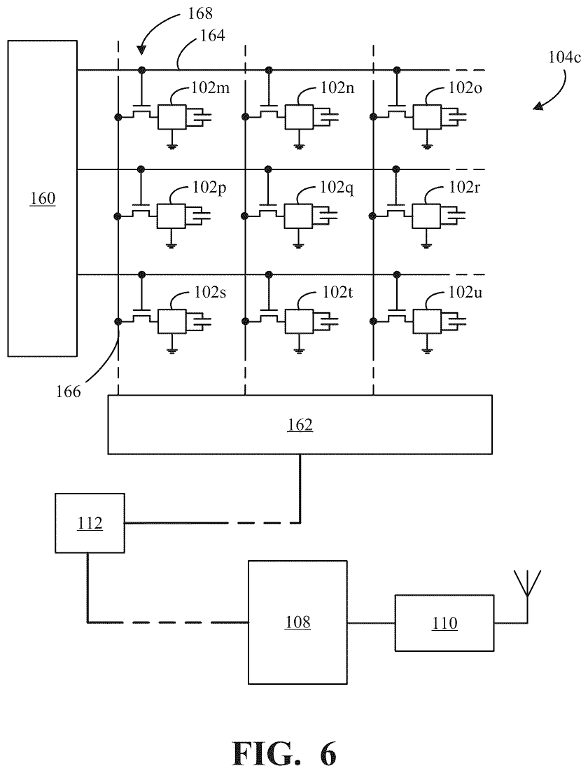

FIG. 6 is an example of a controller (e.g., the controller 108 and/or the local controller 112) that may utilize row/column addressing to read digital data from sensors 102m-u of a sensor array 104c. For example, the sensor array 104c could be another of the plurality of sensor arrays 104 shown in FIG. 1. In the sensor array 104c, the controller can utilize gate drivers 160 and column readout circuitry 162 connected to drive transistors coupled with the sensors 102m-u in matrix to address the sensors 102m-u by row and column. For example, the controller could target sensor 102m in the array for readout by activating row 164 via the gate drivers 160 (e.g., a digital input triggering the sensor via drive transistor 168) and utilizing the column readout circuitry 162 to read column 166 (e.g., the digital output, sent via the drive transistor 168). As a result, in some cases, the controller can perform a parallel readout of sensors in a sensor array. In some implementations, the drive transistors may be implemented by thin film transistors (TFT) in a separate layer coupled with the sensors 102.

Thus, based on the sensors, wiring, and/or configurations of the sensing system 100 as described herein, the controller (e.g., the controller 108 and/or the local controller 112) can target one or more sensors 102 in a sensor array 104 to read digital data from the one or more sensors 102 relatively fast and with high resolution. To enhance speed and/or efficiency, in some cases, the controller can receive a portion of the digital outputs from sensors 102 in a read cycle and may perform multiple read cycles to obtain an entirety of the digital data (e.g., a read rate of 60 Hz). For example, each sensor 102 may include an 8-bit ADC that generates 8 bits of digital data per sampling (e.g., the digital output). The controller can receive one bit of the digital output from each sensor 102 during one read cycle and may perform eight read cycles to receive the complete 8-bits of the digital output from each sensor 102.

In some implementations, the controller (e.g., the controller 108 and/or the local controller 112) may bypass a sensor 102 of a sensor array 104 during a read cycle based on detecting a failure of the sensor 102. For example, the controller can detect that a sensor 102 may be unreliable based on a same or stuck value repeating from the sensor 102 over time. The controller may then bypass reading of the sensor 102 to further improve readout time. In some cases, the electrical interconnect 107 may include bypass circuitry and/or logic to bypass a sensor 102 when the sensor 102 is determined to be unreliable. In some cases, the sensing system 100 may include back up or spare sensors 102 to replace sensors 102 that are determined to be unreliable. For example, in the sensor array 104a, sensor 102b could be a spare sensor that is bypassed if sensor 102a operates correctly or is read by the controller if sensor 102a fails.

In some implementations, the controller (e.g., the controller 108 and/or the local controller 112) may drive an actuation signal into the digital output of a sensor 102 to actuate the sensor area 124 of the sensor. Thus, in some cases, the controller can operate the sensor array 104 bidirectionally so that a sensor 102 can send a digital output based on sensing at the sensor area 124 at one time and receive a digital input (e.g., via the same digital output line) to drive the sensor area 124 at another time. This may be useful, for example, when the sensor area 124 comprises a piezoelectric sensor. The controller can utilize the piezoelectric sensor to perform normal and/or shear force sensing at one time by reading the sensor. The controller can then utilize the piezoelectric sensor to perform proximity sensing at another time by driving a signal to the sensor.

In some implementations, the flexible circuit 128 may include strain relief cutouts between the sensors 102 and the electrical interconnect 107. For example, referring again to FIG. 3, the flexible circuit 128 may include strain relief cutouts 172. The strain relief cutouts 172 may be physical absences of material in the deformable article 106, e.g., the sensing glove, to accommodate flex and strain of the article 106 when worn by the user. For example, the sensor array 104 may be a deformable sensor array that deforms with the article 106.

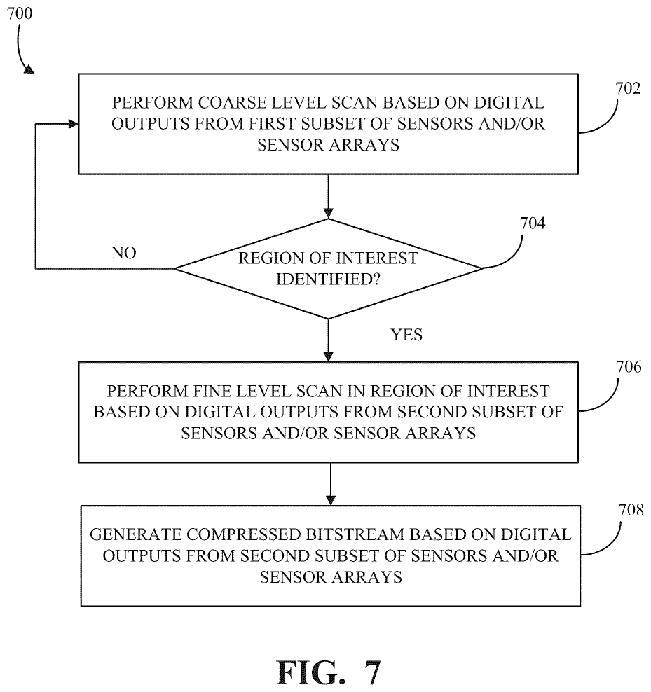

In some implementations, the controller can perform a coarse level read of sensors 102 of the sensing system 100 followed by a fine level read of sensors 102 of the sensing system 100. For example, the controller can utilize fewer sensors 102 to quickly evaluate an environment during the coarse level read, then utilize more sensors 102 to obtain detailed information from the environment in a region of interest during the fine level read. FIG. 7 is a flowchart of a process 700 for reading sensors 102 of the sensor array 104 based on coarse level and fine level reads or scans. The process 700 can be executed using computing devices, such as the systems, hardware, and software described with respect to FIGS. 1-7. The process 700 can be performed, for example, by executing a machine-readable program or other computer-executable instructions, such as routines, instructions, programs, or other code. The operations of the process 700 or another technique, method, process, or algorithm described in connection with the implementations disclosed herein can be implemented directly in hardware, firmware, software executed by hardware, circuitry, or a combination thereof.

For simplicity of explanation, the process 700 is depicted and described herein as a series of operations. However, the operations in accordance with this disclosure can occur in various orders and/or concurrently. Additionally, other operations not presented and described herein may be used. Furthermore, not all illustrated operations may be required to implement a technique in accordance with the disclosed subject matter.

At operation 702, the controller may perform a coarse level read or scan utilizing digital outputs from a first subset of sensors 102 and/or sensor arrays 104. For example, the coarse level scan could comprise reading every other sensor 102 and/or every other sensor array 104 of the sensing system 100. The controller can receive the digital outputs from sensors of the first subset when performing the coarse level scan. In some implementations, the controller may be a global controller utilizing a first subset of local controllers to perform the coarse level scan.

At operation 704, the controller may determine whether a region of interest has been identified. For example, a region of interest may be identified based on detecting an object exerting a normal and/or shear force at a given sensor 102 or a given sensor array 104. In other cases, the region may be identified based on detecting an object in proximity and/or a temperature change. If a region is not identified (“No”), the controller may return to operation 702 to continue a next coarse level scan at a given frequency (e.g., 60 Hz). However, if a region is identified (“Yes”), at operation 706, the controller may perform a fine level read or scan in the region of interest utilizing digital outputs from a second subset of sensors 102 and/or sensor arrays 104. For example, the fine level scan could comprise reading every sensor 102 of a sensor array 104 and/or every sensor array 104 of a section, (e.g., the first finger or fingertip of the sensing glove). The controller can receive the digital output from the second subset of sensors 102 when performing the fine level scan in the region. In some implementations, the controller may be a global controller utilizing a second subset of local controllers to perform the fine level scan.

At operation 708, the controller can generate a compressed bitstream based on digital outputs from the second subset of sensors 102 and/or sensor arrays 104 when performing the fine level scan. The controller can then send the compressed bitstream to another system. For example, the controller could send the compressed bitstream to a host computer or server. In another example, the controller could be a local controller that sends the compressed bitstream to a global controller.

As used herein, the term “circuitry” refers to an arrangement of electronic components (e.g., transistors, resistors, capacitors, and/or inductors) that is structured to implement one or more functions. For example, a circuit may include one or more transistors interconnected to form logic gates that collectively implement a logical function. While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A sensing system, comprising:
a sensor array including a plurality of sensors coupled with a flexible circuit, each sensor including a sensing element and an analog-to-digital converter die coupled with the sensing element, the sensing element including a sensor area coupled with a sensor die to generate an analog output based on sensing, and the analog-to-digital converter die including circuitry to perform analog-to-digital conversion of the analog output to generate a digital output; and
a controller connected to the sensor array, the controller causing a sensor of the sensor array to transmit a digital output that indicates sensing by the sensor in response to receiving a digital input.

2. The sensing system of claim 1, wherein the controller utilizes a serial bus connected with sensors of the sensor array to send a digital input to a sensor and receive a digital output from a sensor.

3. The sensing system of claim 1, wherein the controller causes sensors of the sensor array to sequentially transmit a digital output.

4. The sensing system of claim 1, wherein sensors of the sensor array receive the digital input directly from the controller to cause transmission of a digital output.

5. The sensing system of claim 1, wherein a downstream sensor of the sensor array receives a digital input from an upstream sensor of the sensor array to cause transmission of a digital output.

6. The sensing system of claim 1, wherein the controller addresses sensors of the sensor array by row and column to cause transmission of a digital output.

7. The sensing system of claim 1, wherein the flexible circuit includes circuitry that defines addresses of sensors of the sensor array.

8. The sensing system of claim 1, wherein sensors of the sensor array are programmed to define their addresses in the sensor array.

9. The sensing system of claim 1, wherein the controller receives a portion of the digital output from sensors of the sensor array in one read cycle.

10. The sensing system of claim 1, wherein the controller receives digital outputs from a subset of sensors of the sensor array to perform a coarse level scan.

11. The sensing system of claim 1, wherein the controller receives digital outputs from a subset of sensors of the sensor array to perform a fine level scan in a region of interest.

12. The sensing system of claim 1, wherein the controller bypasses a sensor of the sensor array during a read cycle based on detecting a failure of the sensor.

13. The sensing system of claim 1, wherein the controller is a local controller that generates a compressed bitstream for a global controller based on digital outputs from one or more sensors of the sensor array.

14. The sensing system of claim 1, wherein the sensor array is one of a plurality of sensor arrays coupled with a plurality of flexible circuits, and wherein the controller is one of a plurality of controllers that process digital outputs for a global controller.

15. The sensing system of claim 1, wherein the sensor area comprises a piezoelectric or capacitive sensor, and wherein the analog-to-digital converter die includes a charge amplifier to amplify the analog output.

16. A wearable system, comprising:
an article to be worn by a user;
a flexible circuit coupled with the article;
a sensor array including a plurality of sensors, each sensor including a sensing element and an analog-to-digital converter die coupled with the sensing element, the sensing element including a sensor area coupled with a sensor die to generate an analog output based on sensing, and the analog-to-digital converter die including circuitry to perform analog-to-digital conversion of the analog output to generate a digital output; and
a controller connected to the sensor array, the controller causing a sensor of the sensor array to transmit a digital output that indicates sensing by the sensor in response to receiving a digital input.

17. The wearable system of claim 16, wherein the article is a sensing glove, and wherein a plurality of sensor arrays is coupled with a plurality of flexible circuits with each sensor array of the plurality of sensor arrays corresponding to a finger of the sensing glove.

18. The wearable system of claim 16, wherein the flexible circuit includes electrical interconnect coupled with sensors of the sensor array and strain relief cutouts between the sensors and the electrical interconnect to enable the sensor array to deform with the article.

19. The wearable system of claim 16, wherein sensors of the sensor array each include a one-time programmable (OTP) register that is programmed to define an address in the sensor array.

20. The wearable system of claim 16, wherein the flexible circuit includes fuses that are trimmed to define addresses of sensors in the sensor array.
